(19) United States
(12) Patent Application Publication
(10) Pub. No.: US 2004/0138258 A1
(43) Pub. Date: Jul. 15, 2004

(54) Inhibitors of 11beta-hydroxysteroid dehydrogenase and uses therefor

(76) Inventor: Hartmut M. Hanauske-Abel, Edge Water, NJ (US)

Correspondence Address: Nixon Peabody LLP, Clinton Square, P.O. Box 31051, Rochester, NY, 14603-1051, US

(21) Appl. No.: 10/667,177

(22) Filed: Sep. 18, 2003

Related U.S. Application Data
(60) Provisional application No. 60/411,622, filed on Sep. 18, 2002.

(51) Int. Cl. A61K 31/445; A61K 31/382; A61K 31/366

(52) U.S. Cl. 514/315; 514/331; 514/432; 514/460

(57) ABSTRACT

The present invention identifies natural and man-made inhibitors of the 11β-hydroxysteroid dehydrogenase enzymes that modulate glucocorticoid bioactivity in vivo by catalyzing either the formation of cortisol by reduction of cortisone, or the removal of cortisol by oxidation to cortisone. This mode of modulating glucocorticoid bioactivity in vivo is significant for inflammatory and allergic conditions, cancer, and several metabolic syndromes.

The present invention claims benefit of U.S. Provisional Application Serial No. 60/411,622, filed Sep. 18, 2002, which is hereby incorporated by reference in its entirety. The subject matter of this application was made, in part, with support from the United Stated Government under Grant No. NIH HD00072 from the National Institutes of Health. The United States Government may retain certain rights.

### FIELD OF THE INVENTION

The present invention relates to natural and man-made inhibitors of the 11β-hydroxysteroid dehydrogenase enzymes that modulate glucocorticoid bioactivity in vivo by catalyzing either the formation of cortisol by reduction of cortisone, or the removal of cortisol by oxidation to cortisone. The inhibitors of the present invention can be used to treat inflammatory and allergic conditions, cancer, and several metabolic syndromes.

### BACKGROUND OF THE INVENTION

Cortisol, the major glucocorticoid (GC), is one of the most significant hormones in the human body. It is involved in major aspects of human health and disease, and there is hardly a biochemical pathway not linked to its actions, directly or indirectly.

Cortisol, a steroid hormone, is synthesized de novo in the adrenal gland and secreted into the bloodstream, from where it reaches all the tissues and cells of the human body. This fact does not imply that all tissues and cells see the same amount of cortisol, however, nor does it imply that cortisol can become biologically available by de novo synthesis only.

The actual bioavailability of GCs like cortisol at the level of individual cells is not merely a function of their concentration in blood, nor of their binding to plasma proteins, nor of the varying densities of steroid hormone receptors in target tissues. The actual bioavailability and bioactivity of GCs, even at the level of individual hormone receptors inside of a cell, is controlled by pre-receptor metabolism of the GCs, mediated by cell and tissue specific enzymes that locally activate or deactivate GCs. Consequently, tissue GC levels do not merely reflect plasma GC levels. The 11β-hydroxysteroid dehydrogenases (11β HSDs) are the most prominent enzymes functioning in pre-receptor metabolism. As such, they play a critical role in determining the local levels of GC and thus its ability to activate GC receptors and hence, modulate target gene transcription and expression. The 11β HSD activity occurs in two isoforms, 11β HSD1 and 11β HSD2, the former encoded on chromosome 1, the latter on chromosome 16.

11β HSD1 catalyses, when tested in vitro, either: i) the inactivation of cortisol, by oxidation of the 11130H group using NADP⁺ as cofactor and generating biologically inert cortisone; or ii) the generation of cortisol, by reduction of the 11 keto group of cortisone using NADPH as cofactor. In vivo, this isoform prefers the reductase direction, i.e. mediates the reactivation of cortisol and similar GCs from their bio-inert 11-keto analogs, rather than being involved in the inactivation of GCs (see Seckl et al., Endocrinology 142:1371-1376 (2001) and references therein). The isoform, which appears to function optimally at cortisol/cortisone concentrations in the μm range, is an efficient local amplifier of GC bioavailability since the inactive 11-ketosteroids, in humans particularly cortisone, are systemically available in high concentrations, i.e. at levels equal to or exceeding those of systemic cortisol. The intracellular reactivation of inert keto substrate by the predominant 11β reductase activity of 11βHSD1 has been shown to occur in many tissues and to affect markedly the local activation of glucocorticoid receptors and thus, of the genes controlled by them. For instance in liver, where GCs oppose the actions of insulin, impaired activity of 11βHSD1 and thus impaired intracellular glucocorticoid regeneration, causes an increase in hepatic insulin sensitivity (Walker et al., J. Clin. Endocrinol. Metab. 80:3155-3159 (1995) and references therein). In the brain, the lack of 11β HSD1 activity ameliorates age-related learning impairments, and selective inhibitors of 11β HSD1 have been proposed to be useful agents for preventing glucocorticoid-associated learning deficits (Yau et al., Proc. Natl. Acad. USA 98:4716-4721 (2001) and references therein). Local modification of GC bioactivity by 11β HSD1 has also been established to occur in numerous other tissues, exemplified by, but not limited to, blood vessel wall, ovary, eye, lymph node, and lung (Stewart et al., Vitam. Horm. 57:249-324 (1999) and references therein; Seckl et al., Endocrinology 142:1371-1376 (2001) and references therein). Since 11β HSD1 can amplify glucocorticoid target gene expression in key sites that control metabolic fuel utilization, enhanced 11β HSD1 activity is important in increasing local glucocorticoid action and promoting adverse metabolic effects. Tissue-specific patterns of 11β HSD1 deregulation are now well recognized and appear to be causal for major human ailments, exemplified by the metabolic syndrome and by human obesity, in which a substantial enhancement of 11β HSD1 activity in adipose tissue has been documented that suffices to amplify glucocorticoid action locally (Rask et al. J. Clin. Endocrinol. Metab. 87:3330-3336 (2002) and references therein). Consequently, inhibition of adipose 11β HSD1 was identified as an exciting target for future drug treatment that aims at reducing GC effects in fat by limiting tissue-specific GC reactivation.

11β HSD2 catalyses exclusively the inactivation of GCs like cortisol, from which it generates cortisone by oxidizing the 11β OH group to an 11 keto group. 11β HSD2 only uses NAD as cofactor. This isoform can function as a dehydrogenase even at cortisol concentrations in the nm range. In this way, 11β HSD2 provides a highly efficient, constitutive barrier against GC access to steroid hormone receptors that interact with either GCs or mineralocorticoids (MCs), mediating their respective biological effects. The shielding of the mineralocorticoid receptor (MR) is particularly important, since GC and MC bind to this receptor with equally high affinity in vitro, whereas in vivo only MCs are able to activate the MR, despite the 100- to 1000-fold excess of GCs. Thus, by local pre-receptor removal of GCs 11β HSD2 protects the MR from illicit activation. Mutations of 11β HSD2 in man and mice have established that in case of a genetic deficiency, the loss of receptor protection leads to a severe condition. GCs are now able to overwhelm and over-activate the MR, particularly in the kidney, and in this way cause the Syndrome of Apparent Mineralocorticoid Excess (SAME), which is characterized by hypertension, hypematremia, hypokalemia, and other potentially life-threatening abnormalities (Holmes et al., Mol. Cell Endocrinol. 171:15-20 (2001) and references therein). 11β HSD2 is expressed in a stringent manner in all mineralocorticoid tissues, including the kidneys. 11β HSD2-catalyzed GC inactivation also has significant biological roles in various other tissues. Of note, expression of 11β HSD2 is also able to affect the function of the GC receptor and consequently, the expression of GC-controlled genes. For instance, the significantly increased expression and activity of 11β HSD2 in cancer cells is actually further induced by GCs of endogenous or exogenous origin, which accelerate their intracellular catabolism and impair their own antiproliferative effect. In this way, 11β HSD2 provides an enzymatic shield that protects these malignantly proliferating cells, e.g. of breast origin, from the antiproliferative effects of GC. Consequently, inhibition of the inactivating 11β HSD2 should markedly enhance the antiproliferative activity of GC. Used jointly with the licorice-derived compound glycyrrhetinic acid as experimental 11β HSD2 inhibitors, GCs were found to indeed inhibit cancer cell proliferation, suggesting inhibition of 11β HSD2 activity in tumor cells retards tumor growth by locally increasing the bioavailability of GCs, whether of endogenous or exogenous origin (Hundertmark et al., J. Endocrinol. 155:171-180 (1997) and references therein). Similarly, the anti-inflammatory activity of GCs can be markedly enhanced by combination with the experimental 11β HSD2 inhibitor glycyrrhetinic acid, as shown for contact hypersensitivity of skin using topical application of the compound combination (Hennebold et al., Arch. Dermatol. Res. 290:413-419 (1998) and references therein).

However, the existing 11β HSD inhibitors like glycyrrhetinic acid are known to be only partial inhibitors of 11β HSD activity. In light of the promising potential as enhancers of cancer sensitivity to the antiproliferative action of GCs, it was therefore concluded that “it is necessary to develop stronger inhibitors of 11β HSD in order to improve the antiproliferative effect of low-dose glucocorticoids” (Hundertmark et al., J. Endocrinol. 155:171-180 (1997) at page 178). These authors also pointed out that increased mineralocorticoid effects due to 11β HSD inhibition, and thus a pharmacological equivalent of SAME, could be easily managed by diuretics and aldosterone antagonists.

The present invention is directed to overcoming these and other deficiencies in the art.

### SUMMARY OF THE INVENTION

The present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows:

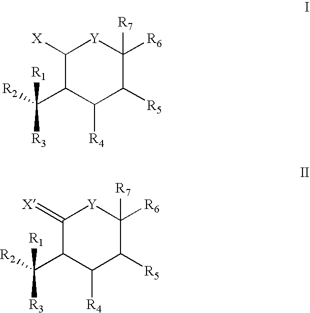

wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R is H, CH₃, CH₂CH₃, or CH₂CH₂CH₃, R₇ is H or CH₃, X is OH, SH, or NH₂, X′ is O, S, or NH, and Y is O, S, NH, or CH₂.

The present invention also relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows:

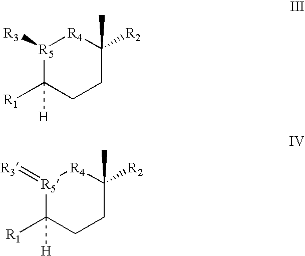

wherein R₁ is

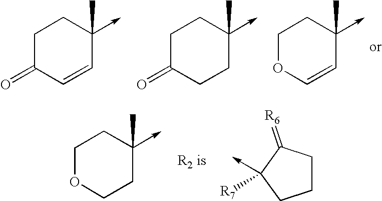

wherein R₆ is O or S and R₇ is H, OH, or halogen, or

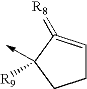

wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂.

Another aspect of the present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows:

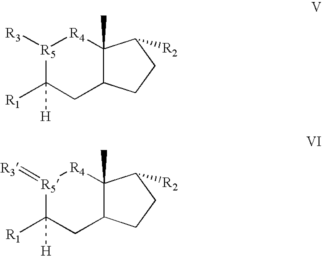

wherein R₁ is

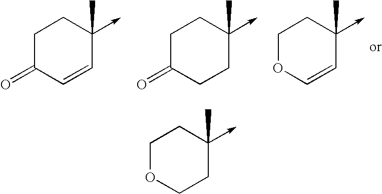

R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

Yet another aspect of the present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows:

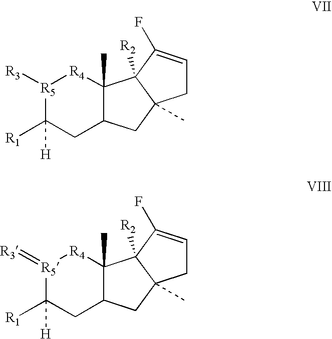

wherein R₁ is

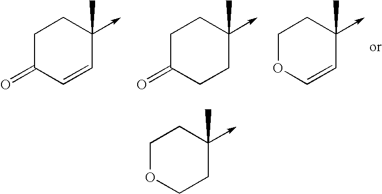

R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

Additional aspects of the present invention relate methods of treating an inflammatory or allergic condition, cancer, obesity, diabetes mellitus, or a metabolic syndrome involving 11β-hydroxysteroid dehydrogenase in a living system. These methods involve administering to the living system an inhibitor of cortisol-to-cortisone conversion, as mediated by 11β-hydroxysteroid dehydrogenase, of formula I, III, V, or VII, or an inhibitor of cortisone-to-cortisol conversion, as mediated by 11β-hydroxysteroid dehydrogenase, of formula II, IV, VI, or VIII, or derivatives thereof as described above under conditions effective to treat an inflammatory or allergic condition, cancer, obesity, diabetes mellitus, or a metabolic syndrome involving 11β-hydroxysteroid dehydrogenase.

In accordance with the present invention, optimized inhibitors of 11β HSD have been identified based on the identification of the minimally required structure for interaction with 11β HSD. As described above, these inhibitors can be used in methods of treating an inflammatory or allergic condition, cancer, obesity, diabetes mellitus, or a metabolic syndrome involving 11β-hydroxysteroid dehydrogenase in a living system.

### BRIEF DESCRIPTION OF THE DRAWINGS

FIG. 1 shows the steric structure of two 11β HSD substrates, the glucocorticoids cortisol and corticosterone, and two isomers of menthol, (1S, 2S, 5R)-neomenthol and (1S, 2R, 5R)-isomenthol. Note the sterically distinct, axial vs. equatorial substitution at C2 of the menthol isomers. Only neomenthol mirrors precisely onto the glucocorticoids, extending from ring A over ring B to ring D, with a complete fit at ring C. Neomenthol exactly mimics the steric arrangement at carbon atoms 1, 10, 19, 9, 11, 12, 13, and 17.

FIG. 2 shows the computational analysis and visualization of cortisol [I], (1S, 2S, 5R)-neomenthol [II] and (1S, 2R, 5R)-isomenthol [III]—axial line of view. The white line bracket highlights the area of homology, centering on the oxygen atom at C 11, the subject of the redox activity of 11β HSD. An arrow indicates the relevant proton. *, position of the C19 methyl moiety or equivalent; white arrow, substrate proton of 11β HSDs; solid isosurfaces: 0.08 electrons/au³; dotted isosurfaces, 0.002 electrons/au³; electrostatic potential gray-shaded onto each isosurface. Note the marked difference between neomenthol and isomenthol when compared with cortisol.

FIG. 3 shows the computational analysis and visualization of cortisol [I], (1S, 2S, 5R)-neomenthol [II] and (1S, 2R, 5R)-isomenthol [III]—equatorial line of view. The white line bracket highlights the area of homology, centering on the oxygen atom at C 11, the subject of the redox activity of 11β HSD. An arrow indicates the relevant proton. *, position of the C19 methyl moiety or equivalent; white arrow, substrate proton of 11β HSDs; solid isosurfaces: 0.08 electrons/au³; dotted isosurfaces, 0.002 electrons/au³; electrostatic potential gray-shaded onto each isosurface. Note the marked difference between neomenthol and isomenthol when compared with cortisol.

FIG. 4 is a Lineweaver-Burk plot, and its secondary plot, of corticosterone utilization by rat liver 11β HSD (oxidative activity) in the presence of increasing concentrations of (1S, 2S, 5R)-(+) neomenthol. The concentrations of the inhibitor are indicated. The mode of inhibition is competitive, indicating that this menthol isomer binds precisely like the GC substrate to 11β HSD (see 84^{th} Annual Meeting, Endocrine Society; p273 (#P1-516) (2002)).

FIG. 5 is a Lineweaver-Burk plot, and its secondary plot, of corticosterone utilization by rat liver 11β HSD (oxidative activity) in the presence of increasing concentrations of (1S, 2R, 5R)-isomenthol. The concentrations of the inhibitor are indicated. The mode of inhibition is non-competitive, indicating that this menthol isomer does not bind like the GC substrate to 11β HSD (see 84^{th} Annual Meeting, Endocrine Society; p273 (#P1-516) (2002)).

FIG. 6 shows the structure of the 11β HSD substrates cortisol and cortisone, and the small molecule conformation homologues, (1S, 2S, 5R)-neomenthol and (2R, 5R)-neomenthone, respectively. Note that the monoterpene conformation homologues mirrors precisely onto cortisol and cortisone, extending from ring A over ring B to ring D, with a complete fit at ring C. Their conformation therefore makes them suitable lead compounds for improved small molecule inhibitors that modulate in vivo the activity of 11β HSD2 and of 11β HSD1, respectively.

FIG. 7 shows the structural formulae (top and bottom), and computationally generated composite surfaces (left, isodensity surface at 0.08 electrons/au³; middle, isodensity surface at 0.002 electrons/au³; right, ball-and-stick model; electrostatic potential gray-shaded onto each isosurface; fine dots, electrostatic potential cloud at 20 kcal/mol) for cortisol (compound I) and for neomenthol (compound II).

FIG. 8 shows structural formulae (top and bottom), and computationally generated composite surfaces (left, isodensity surface at 0.08 electrons/au³; middle, isodensity surface at 0.002 electrons/au³; right, ball-and-stick model; electrostatic potential gray-shaded onto each isosurface; fine dots, electrostatic potential cloud at 20 kcal/mol) for cortisol (compound I) and for compound III.

FIG. 9 shows structural formulae (top and bottom), and computationally generated composite surfaces (left, isodensity surface at 0.08 electrons/au³; middle, isodensity surface at 0.002 electrons/au³; right, ball-and-stick model; electrostatic potential gray-shaded onto each isosurface; fine dots, electrostatic potential cloud at 20 kcal/mol) for cortisol (compound I) and for compound IV.

FIG. 10 shows structural formulae (top and bottom), and computationally generated composite surfaces (left, isodensity surface at 0.08 electrons/au³; middle, isodensity surface at 0.002 electrons/au³; right, ball-and-stick model; electrostatic potential gray-shaded onto each isosurface; fine dots, electrostatic potential cloud at 20 kcal/mol) for cortisol (compound I) and for compound V.

FIG. 11 shows the proposed dual action of neomenthol, facilitating release of plasma protein-bound and therefore biologically unavailable cortisol while at the same time, inhibiting the cortisol-to-cortisone dehydrogenation (cortisol inactivation) mediated by 11β HSD activity. The local effect amounts to an increase in the bioavailable cortisol and thus, to locally enhanced biological effects of cortisol and similar glucocorticoids (see 84^{th} Annual Meeting, Endocrine Society; p273 (#P1-516) (2002)).

### DETAILED DESCRIPTION OF THE INVENTION

The present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows:

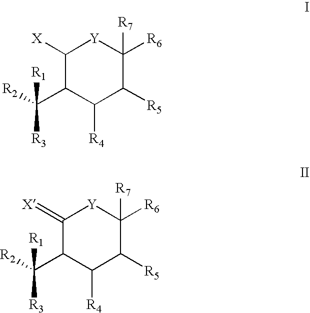

wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R₆ is H, CH₃, CH₂CH₃, or CH₂CH₂CH₃, R₇ is H or CH₃, X is OH, SH, or NH₂, X′ is O, S, or NH, and Y is O, S, NH, or CH₂.

In one embodiment, the inhibitor has the formula:

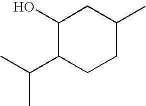

In another embodiment, the inhibitor has the formula:

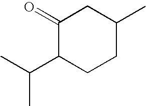

The present invention also relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows:

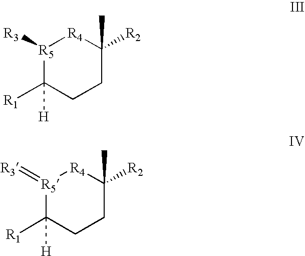

wherein R₁ is

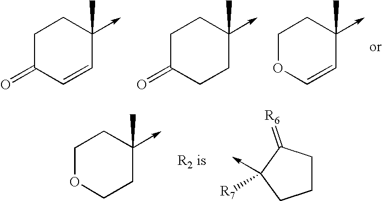

wherein R₆ is O or S and R₇ is H, OH, or halogen, or

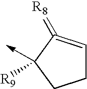

wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂.

Another aspect of the present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows:

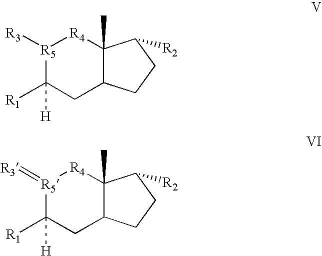

wherein R₁ is

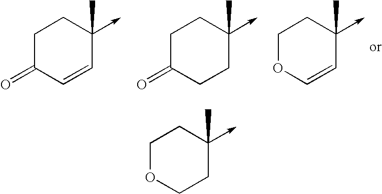

R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

Yet another aspect of the present invention relates to a method of inhibiting 11β-hydroxysteroid dehydrogenase in a living system. This method involves administering to the living system an effective amount of an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows:

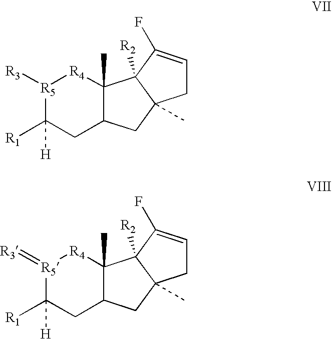

wherein R₁ is

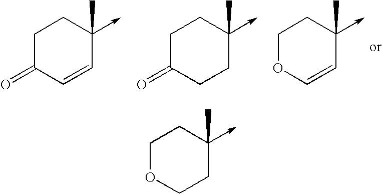

R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

In accordance with one embodiment, the inhibitors of the present invention inhibit isoform I of 11β HSD.

In accordance with another embodiment, the inhibitors of the present invention inhibit isoform II of 11β HSD.

As used herein, suitable living systems include, but are not limited to, mammals, including dogs, cats, rats, mice, and humans, and non-mammalian species like fish or insects.

Suitable derivatives of the above-identified inhibitors include, but are not limited to, esters, amides, and their salts.

One of the most productive strategies for the discovery of an enzyme inhibitor, e.g. an antagonist of 11β HSD1 or 11β HSD2, involves the identification of the minimally required structure able to bind to, and thus be a substrate for or an inhibitor of, the enzyme of interest. This is followed by the knowledge-guided formulation of a series of optimized analogs and derivatives, applying structural principles of molecular pharmacology that are known to those trained in the art (see, e.g., Hanauske-Abel et al., Curr. Med. Chem. 10:1005-1019 (2003) for a description of a successful application of this strategy to the discovery of lead compounds for inhibition of enzymes that hydroxylate proteins).

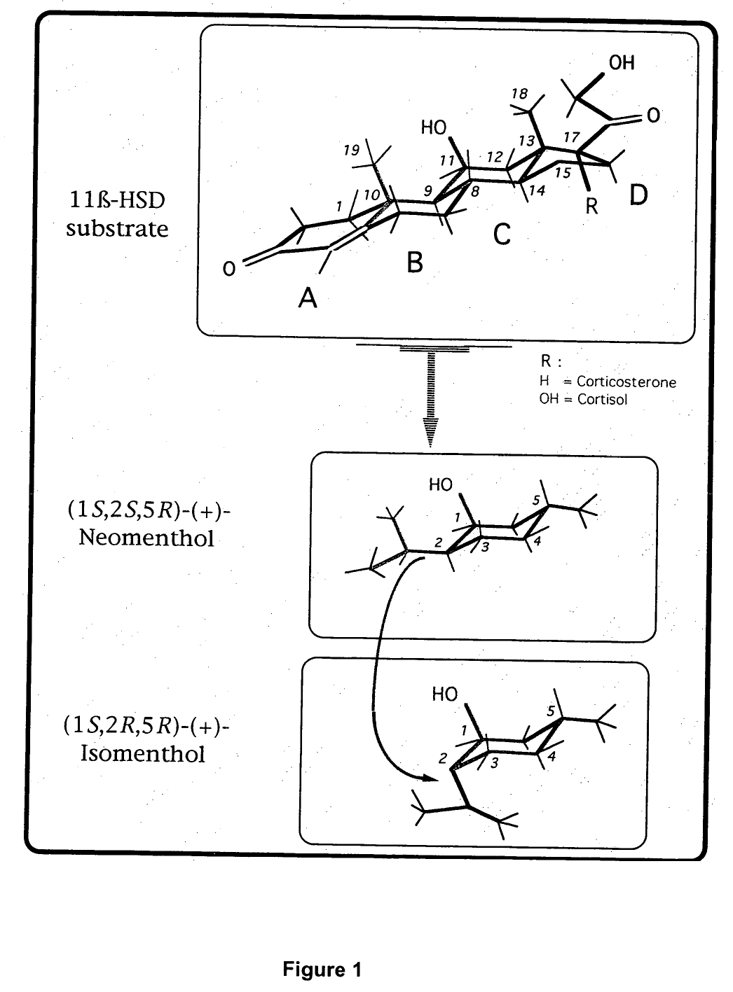

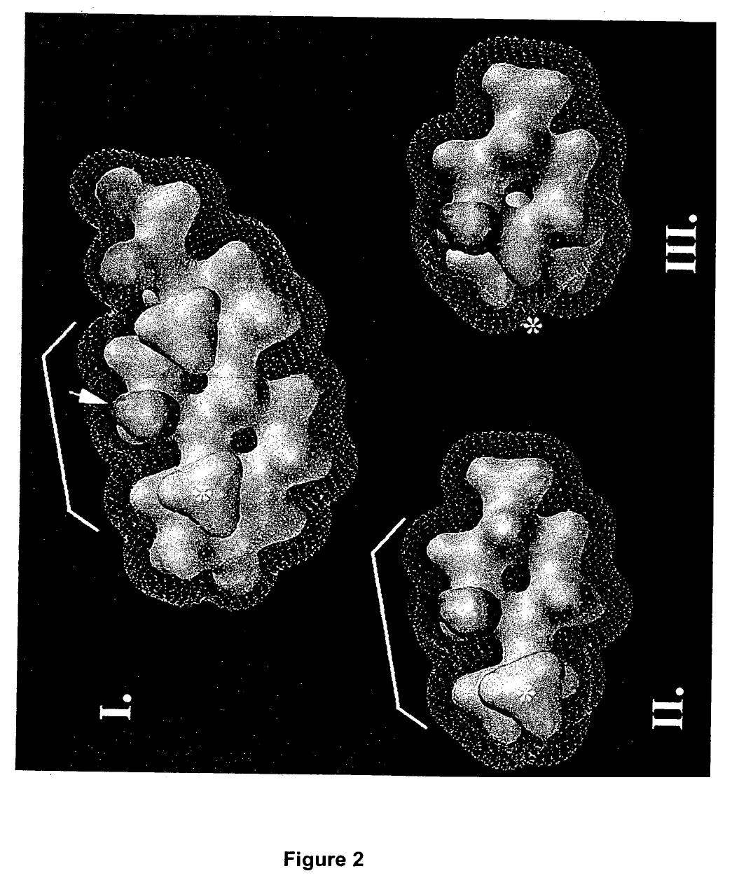

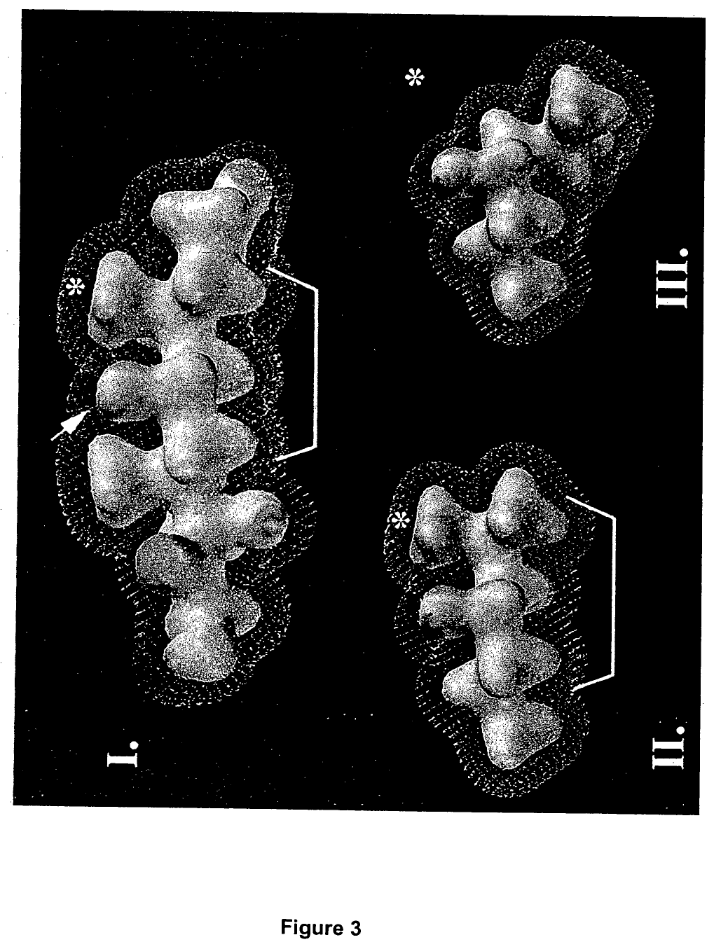

This strategy has been applied to the discovery of lead compounds that inhibit 11β HSD in accordance with the present invention. In particular, the menthol/menthone series of monoterpenes, which comprises a significant number of stereoisomers, can unexpectedly be considered as mimics of a domain in cortisol that centers on the ring C, but also extends to rings A, B, and D (see FIGS. 1-3). As shown in FIGS. 2 and 3, optimal alignment of the carbon skeleton also aligns the position of the oxygen atom at C1 in the menthol/menthone series with the position of the oxygen atom at C11 of cortisol.

This surprising fact indicated that at least some of the menthol/menthone monoterpenes may, with regard to the reductive and the oxidative activities of the 11β HSD isoenzymes, actually function as a minimal fragment of cortisol, the physiological molecule that interacts with the enzyme. This was shown experimentally using the oxidative in vitro activity of 11β HSD from liver. Identified as the minimally required structure for interaction with 11β HSD, menthol/menthone were then used as the structures to develop a series of optimized analogs and derivatives, applying structural principles of molecular pharmacology that are known to those trained in the art. Thus, the above inhibitors of the present invention were identified.

Synthesis of the compounds of the present invention can be achieved using methods known to those of ordinary skill in the art. In particular, the compounds of the present invention can be synthesized retrosynthetically, based on the identification of the optimal structures for inhibitors in accordance with the present invention, as pathways to produce the desired compounds would be obvious to one of ordinary skill in the chemical arts. Synthesis can be carried out either manually or through the use of an automated process. For manual synthesis, the chemical manipulations would be performed by a scientist or technician. For automated synthesis, the chemical manipulations would typically be performed robotically. The choice and implementation of such techniques is within the skill of one of ordinary skill in the chemical arts and will not be discussed in detail herein.

Additional aspects of the present invention relate methods of treating an inflammatory or allergic condition, cancer, obesity, diabetes mellitus, or a metabolic syndrome involving 11β-hydroxysteroid dehydrogenase in a living system. These methods involve administering to the living system an inhibitor of cortisol-to-cortisone conversion, as mediated by 11β-hydroxysteroid dehydrogenase, of formula I, III, V, or VII, or an inhibitor of cortisone-to-cortisol conversion, as mediated by 11β-hydroxysteroid dehydrogenase, of formula II, IV, VI, or VIII, or derivatives thereof as described above under conditions effective to treat an inflammatory or allergic condition, cancer, obesity, diabetes mellitus, or a metabolic syndrome involving 11β-hydroxysteroid dehydrogenase.

As used herein, inflammatory or allergic conditions include, but are not limited to, acute or chronic conditions caused or aggravated by the activation and involvement of humoral and/or cellular elements of the immune system in response to exogenous or endogenous triggers. Examples include dermatological conditions such as hypersensitivity reactions and allergies; pulmonary conditions such as asthma; gastrointestinal conditions such as ulcerative colitis; and systemic conditions such as multiple sclerosis or rheumatoid arthritis; as well as rejection of transplants.

As used herein, metabolic syndromes involving 11β-hydroxysteroid dehydrogenase include, but are not limited to obesity, diabetes mellitus, and the various conditions involving insulin resistance, such as ovarian hyperandrogenism or Syndrome X.

In accordance with the methods of the present invention, the inhibitor of the present invention can be administered alone, or in combination with suitable pharmaceutical carriers or diluents. The diluent or carrier ingredients should be selected so that they do not diminish the therapeutic effects of the inhibitors of the present invention or compositions. Suitable pharmaceutical compositions include those which include a pharmaceutical carrier and, for example, one or more of an inhibitor, as described herein. A pharmaceutically acceptable medium can additionally contain physiologically acceptable compounds that act, for example, to stabilize or increase the absorption of the inhibitor of the present invention, analogue, mimetic, or chemical derivative. Such physiologically acceptable compounds include, for example, carbohydrates such as glucose, sucrose, or dextrans; antioxidants such as ascorbic acid or glutathione; chelating agents such as EDTA, which disrupts microbial membranes; divalent metal ions such as calcium or magnesium; low molecular weight proteins; lipids or liposomes; or other stabilizers or excipients.

The inhibitors of the present invention and compositions herein can be made up in any suitable form appropriate for the desired use; e.g., oral, parenteral, or topical administration. Thus, topical and/or system administration may be used. Examples of parenteral administration are intraventricular, intracerebral, intranasal, intraocular, intramuscular, intravenous, intraarterial, intraperitoneal, by intraversal instillation, intralesion, rectal, and subcutaneous administration. Administration may also be achieved by application to mucous membranes.

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, such as methyl cellulose, tragacanth, sodium alginate; wetting agents, such as lecithin and polyoxyethylene stearate; and preservatives, such as ethyl-p-hydroxybenzoate.

Dosage forms suitable for parenteral administration include solutions, aqueous and non-aqueous suspensions which can include suspending agents and thickening agents, dispersions, emulsions, and the like. They may also be manufactured in the form of sterile solid compositions which can be dissolved or suspended in sterile injectable medium immediately before use. They may contain suspending or dispersing agents known in the art. The solutions, suspensions, dispersions, emulsions, and the like can additionally contain, for example, anti-oxidants, buffers, bacteriostats, and solutes which render the formulation isotonic with the blood of the intended recipient. The formulations can be presented in unit-dose or multi-dose containers, for example, sealed ampoules and vials. Extemporaneous injection solutions and suspensions can be prepared from sterile powders, granules, and tablets of the kind previously described.

For oral administration either solid or fluid unit dosage forms can be prepared. For preparing solid compositions, such as tablets, a suitable inhibitor of the present invention or composition, as disclosed above, is mixed with conventional ingredients, such as talc, magnesium stearate, dicalcium phosphate, magnesium aluminum silicate, calcium sulfate, starch, lactose, acacia methylcellulose, and functionally similar materials as pharmaceutical diluents or carriers. Capsules are prepared by mixing the disclosed inhibitors of the present invention or compositions with an inert pharmaceutical diluent and filling the fixture into a hard gelatin capsule of appropriate size. Soft gelatin capsules are prepared by machine encapsulation of a slurry of the inhibitor of the present invention or composition with an acceptable vegetable oil, light liquid petrolatum, or other inert oil.

Fluid unit dosage forms for oral administration such as syrups, elixirs, and suspensions can be prepared. The water-soluble forms can be dissolved in an aqueous vehicle together with sugar, aromatic flavoring agents, and preservatives to form a syrup. An elixir is prepared by using a hydro-alcoholic (ethanol) vehicle with suitable sweeteners, such as sugar and saccharin, together with an aromatic flavoring agent. Suspensions can be prepared with a syrup vehicle with the aid of a suspending agent, such as acacia, tragacanth, methylcellulose, and the like.

When the inhibitors of the present invention or compositions are administered orally, suitable daily dosages can be based on suitable doses of glucocorticoids, such as those described in Goodman and Gilman, The Pharmacological Basis of Therapeutics, 7^{th} edition, which is hereby incorporated by reference in its entirety. Typically, for oral administration, suitable daily doses are from about 0.5 mg/d to about 60 mg/d of the inhibitor of the present invention for adult patients, with proper adjustments for the spectrum of pediatric patients. Alternatively, the inhibitors of the present invention or compositions can be administered orally in foodstuffs.

For parenteral administration, fluid unit dosage forms are prepared utilizing the aforementioned inhibitors of the present invention or compositions and a sterile vehicle, water being preferred. The inhibitor of the present invention or composition, depending on the vehicle and concentration used, can be either suspended or dissolved in the vehicle. In preparing solutions, the inhibitor of the present invention or composition can be dissolved in water for injection and filter sterilized before filling into a suitable vial or ampule and sealing. Advantageously, adjuvants, such as a local anesthetic, preservative, and buffering agents, can be dissolved in the vehicle. To enhance the stability, the fluid unit dosage form can be frozen after filling into the vial, and the water removed under vacuum. The dry lyophilized powder is then sealed in the vial, and an accompanying vial of water for injection is supplied to reconstitute the liquid prior to use. Parenteral suspensions are prepared in substantially the same manner except that the inhibitor of the present invention or composition is suspended in the vehicle instead of being dissolved, and sterilization cannot be accomplished by filtration. The inhibitor of the present invention or composition can be sterilized by exposure to ethylene oxide before suspending in the sterile vehicle. Advantageously, a surfactant or wetting agent is included in the parenteral suspension to facilitate uniform distribution of the inhibitor of the present invention or composition. Parenteral dosages typically can range from about 0.5 mg/d to about 500 mg/d of the inhibitor of the present invention for adult patients, with proper adjustments for the spectrum of pediatric patients.

Alternatively, the inhibitor of the present invention or composition can be used in polymeric formulations and sustained release formulations and surgically implanted using conventional methods. Suitable sustained release matrices include those made of ethylene vinyl acetate and other biocompatible polymers. The inhibitor of the present invention can be covalently attached by surface grafting or co-polymerization, non-covalently incorporated into a matrix, or otherwise encapsulated as biomedical materials. This is one example of a drug delivery method involving conjugation of the inhibitor of the present invention to a carrier material that can be used to locally deliver the anti-11β-hydroxysteroid dehydrogenase effects of such a formulation.

For topical administration, carriers, such as phospholipid vesicles, which contain the aforementioned inhibitor of the present invention or composition may facilitate uptake through the skin.

All publications mentioned herein are hereby incorporated by reference in their entirety.

The present invention is further illustrated by the following examples.

### EXAMPLES

### Example 1

### Materials and Methods

Computational analyses were performed using the latest version of MacSpartan Plus and MSI InsightII. Experimental analysis employed the rat enzyme as described (Monder et al., BBA 115:23-29 (1991) and references therein). Kinetic data were analyzed in Excel and plotted in CricketGraph.

### Example 2

### Computational Analyses

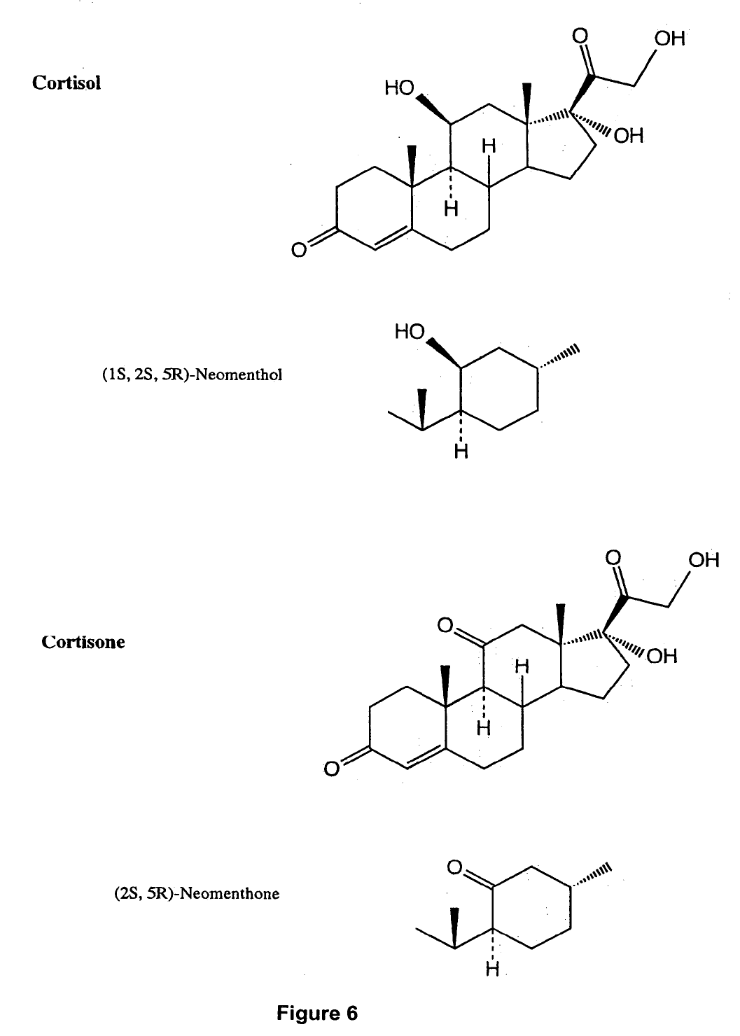

To detect steric and electrostatic homologies between glucocorticoids like cortisol and the monoterpenes that conform with the conformational restrictions that define glucocorticoids, i.e the neomenthols identified in FIG. 6, these molecules were modeled with regard to molecular orbitals, electron densities, spin densities, potentials, and optimal geometry. Molecules of interest were build from the atomic fragment catalog of the Spartan software package (Wavefunction, Irvine, Calif.). Molecular mechanics analysis was performed with both the semi-empirical AMI menu and ab initio Hartree-Fock calculations using the 3-21 G^{(*)} basis set. Quantities resulting from these calculations were graphically displayed to yield an image portraying both the geometrically optimized and the electrostatic characteristics of a given molecule. For each molecule, surface area and volume were determined in Å² and Å³, respectively. Electrostatic charges for the atoms of the domain shared by all compounds, were calculated by Mulliken Population Analysis.

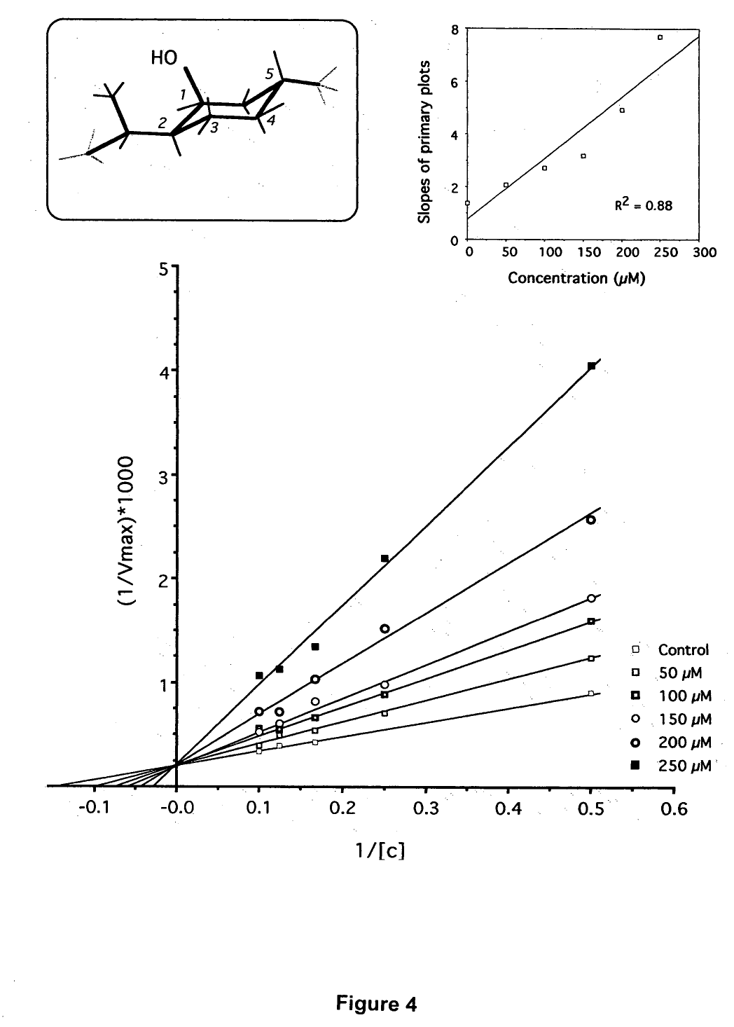

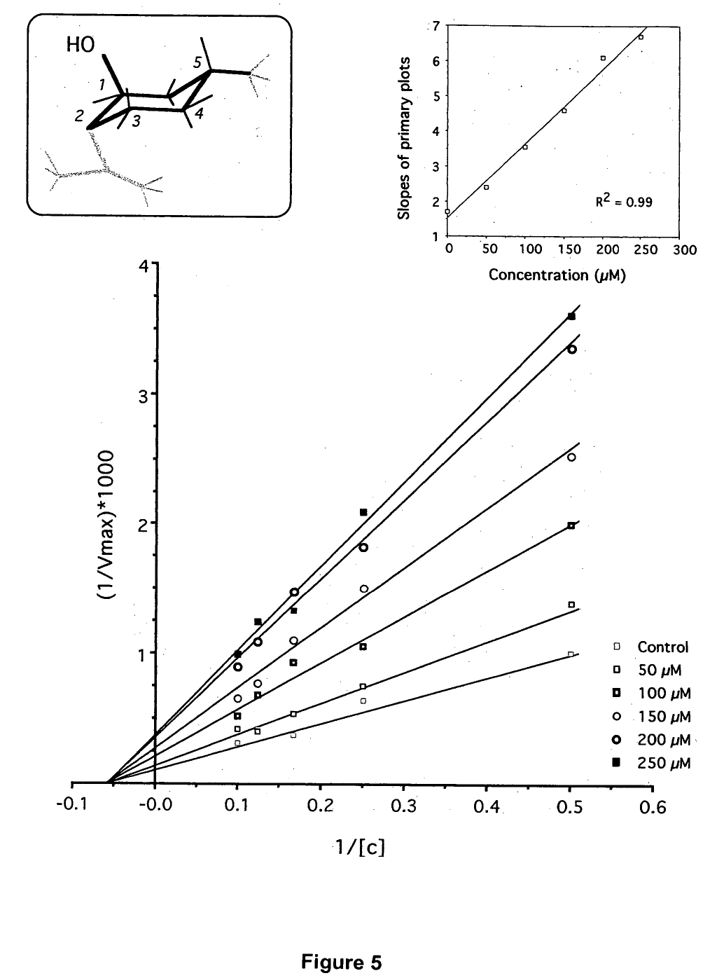

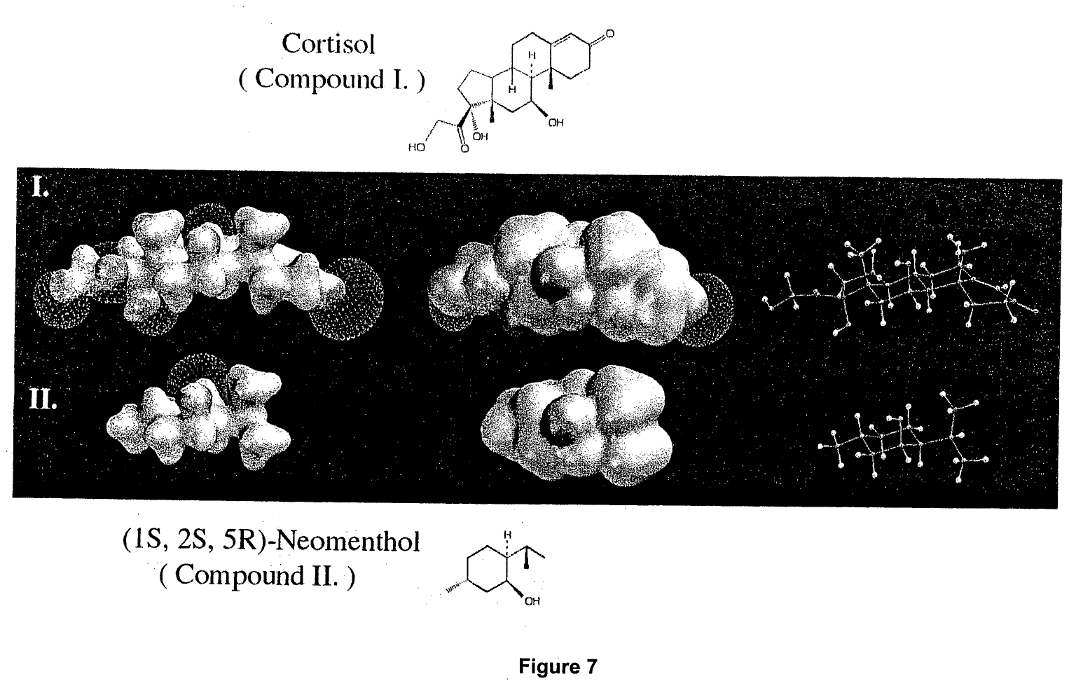

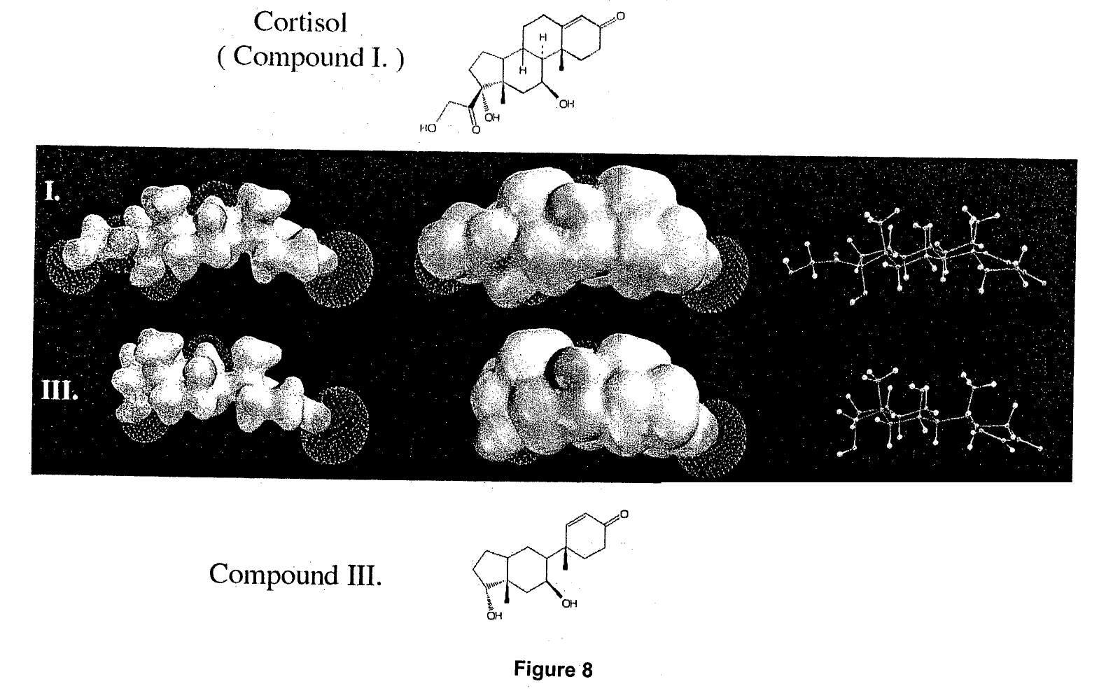

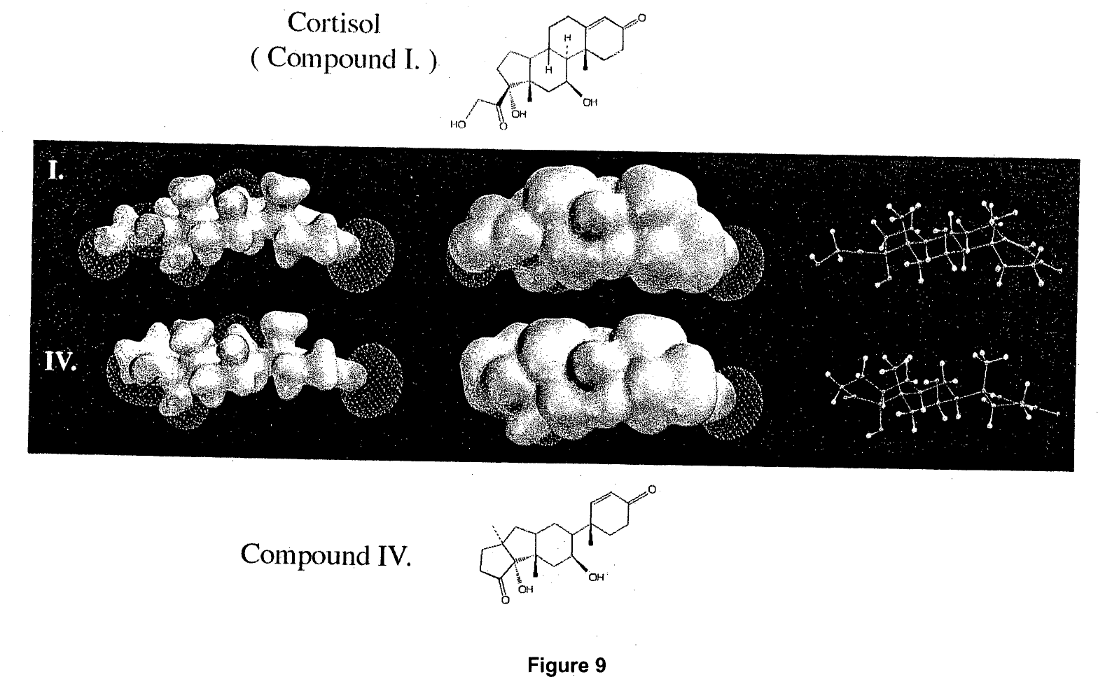

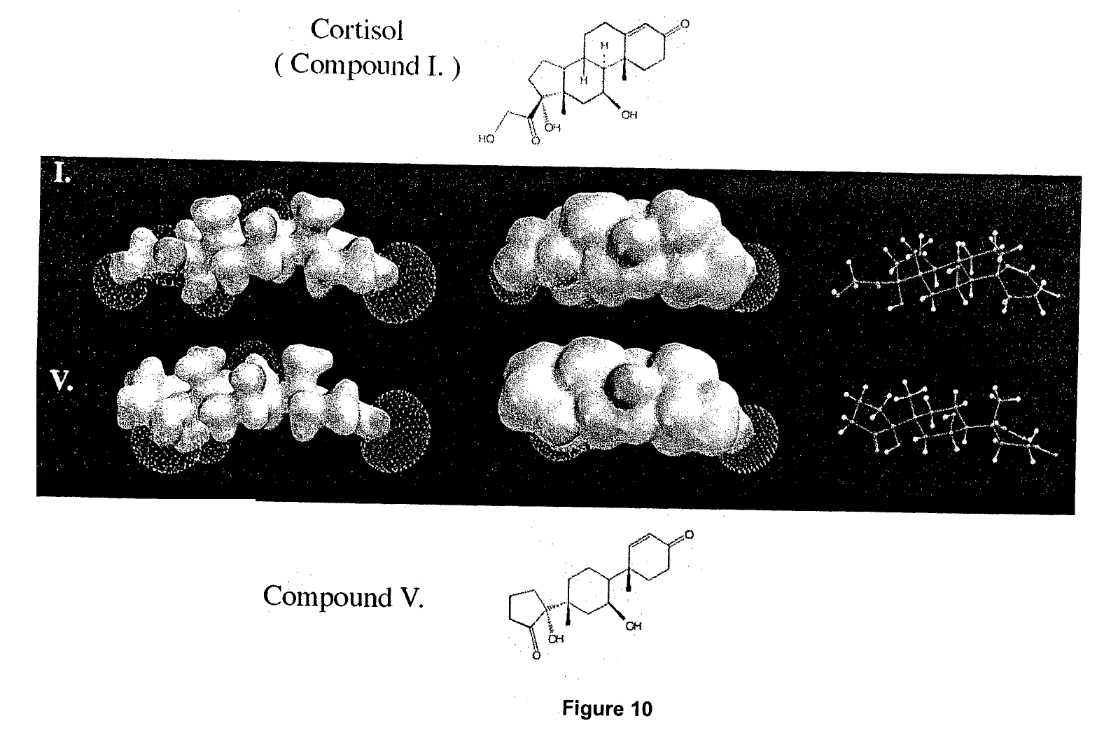

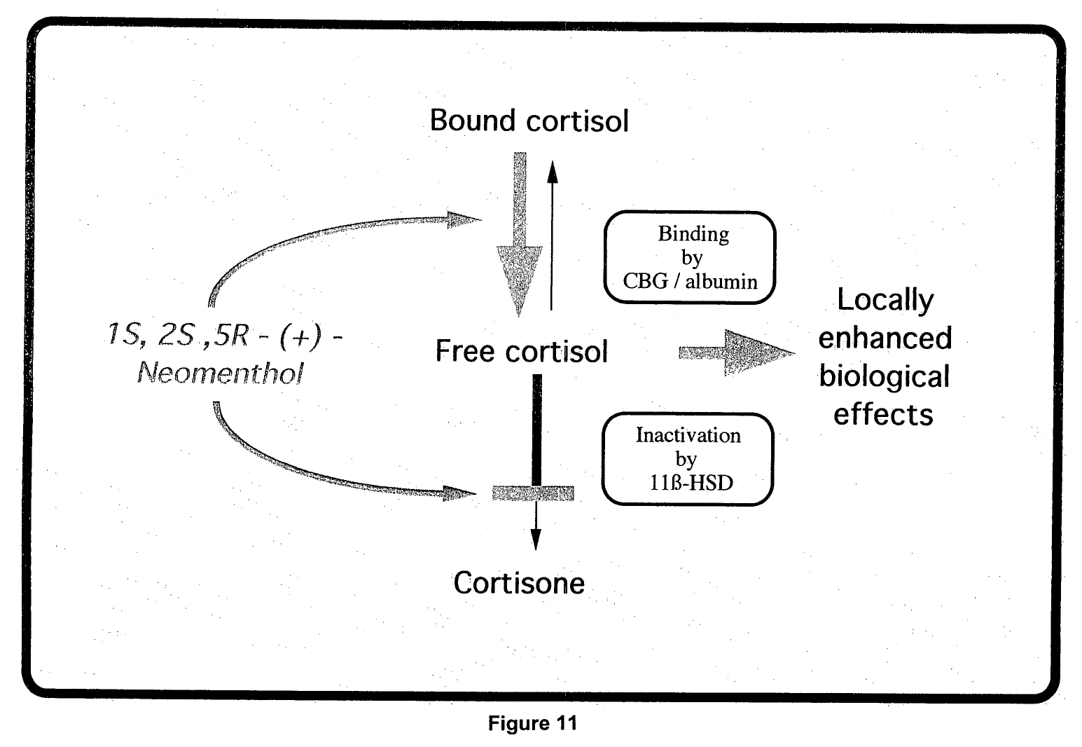

The structural similarity between GCs like cortisol and the monoterpenes with the conformation of neomenthol (FIG. 1) was confirmed by the computational analysis of their steric and electronic similarities, as summarized in FIGS. 2 and 3. Only neomenthol precisely mimicked the region of GCs around C11 that contains the oxygen atom subjected to the redox reactions mediated by the 11β HSDs. Consequently, only neomenthol should bind to such an enzyme like its physiological substrate, competing with said substrate for the active site of said enzyme. This computation-based prediction was confirmed experimentally, as shown in FIGS. 4 and 5 for the 11β HSDs isolated from rat liver. The purified rat liver 11β HSD, when used in vitro, utilizes NADP+ and oxidizes cortisol and corticosterone in an ordered sequential bireactant mechanism (Monder et al., Biochim. Biophys. Acta 1115:23-29 (1991) and references therein). Employing 1,2,6,7-3H-corticosterone, neomenthol, and isomenthol inhibited the enzyme. With regard to the glucocorticoid substrate, however, only neomenthol displayed a competitive mode of inhibition (Kᵢₛ=35 μM) (FIG. 4), whereas isomenthol displayed a non-competitive mode of inhibition (Kᵢₛ=71 μM) (FIG. 5). These results suggested that the widely available over-the-counter mentholated rubefacients may locally enhance the bioavailability of endogenous cortisol. More importantly, the results identified the conformation of neomenthol as a lead for the rational optimization of 11β HSD inhibitors, applicable to both the in vivo oxidizing activity of 11β HSD2 and the in vivo reducing activity of 11β HSD1, as shown in FIG. 6. Using the same computational algorithms that correctly predicted the competitive mode of inhibitory activity for neomenthol, and the lack thereof for isomenthol, molecules were assembled in silico and evaluated for their computed physicochemical characteristics relevant for the desired inhibition of 11β HSD activity. Originating from the lead compounds (FIG. 6), the computational analysis converged on several optimized structures that contained combinations of physicochemical and steric elements prerequisite for 11β HSD inhibition. These structures are identified in FIGS. 7-10. FIG. 7 details for neomenthol the findings obtained with this approach. FIG. 8 details the findings for a representative compound in which, among other modifications, a structure equivalent to the B ring of cortisol has been deleted. FIG. 9 details the findings for a representative compound in which, among other modifications, a ring has been added to the structure equivalent to the D ring of cortisol. FIG. 10 details the findings for a representative compound in which, among other modifications, a structure equivalent to the D ring of cortisol has been deleted. As originally proposed for neomenthol (FIG. 11), these structures are sufficiently isosteric and isoelectronic with cortisol to displace it from its plasma binding proteins, in this way increasing the bioavailable, free cortisol in a living system, e.g. after topical administration to skin only in skin blood vessels.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of inhibiting 11β-hydroxysteroid dehydrogenase in a living system comprising:
administering to the living system an effective amount of an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows: 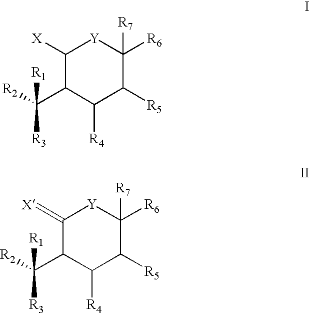
wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R₆ is H, CH₃, CH₂CH₃, or CH₂CH₂CH₃, R₇ is H or CH₃, X is OH, SH, or NH₂, X′ is O, S, or NH, and Y is O, S, NH, or CH₂.

2. The method according to claim 1, wherein the 11β-hydroxysteroid dehydrogenase is isoform I.

3. The method according to claim 1, wherein the 11β-hydroxysteroid dehydrogenase is isoform II.

4. The method according to claim 1, wherein the inhibitor has the formula:

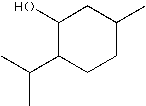

5. The method according to claim 1, wherein the inhibitor has the formula:

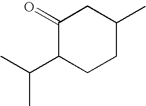

6. The method according to claim 1, wherein the administering is carried out topically.

7. The method according to claim 1, wherein the inhibitor is administered with pharmaceutically acceptable carrier, excipient, or stabilizer.

8. A method of treating an inflammatory or allergic condition in a living system comprising:
administering to the living system an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows: 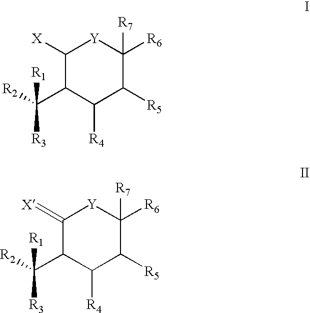
wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R₆ is H, CH₃, CH₂CH₃, or CH₂CH₂CH₃, R₇ is H or CH₃, X is OH, SH, or NH₂, X′ is O, S, or NH, and Y is O, S, NH, or CH₂, under conditions effective to treat an inflammatory or allergic condition.

9. The method according to claim 8, wherein the 11β-hydroxysteroid dehydrogenase is isoform I.

10. The method according to claim 8, wherein the 11β-hydroxysteroid dehydrogenase is isoform II.

11. The method according to claim 8, wherein the inhibitor has the formula:

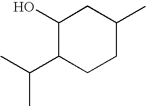

12. The method according to claim 8, wherein the inhibitor has the formula:

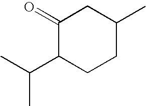

13. The method according to claim 8, wherein the administering is carried out topically.

14. The method according to claim 8, wherein the inhibitor is administered with pharmaceutically acceptable carrier, excipient, or stabilizer.

15. A method of treating cancer in a living system comprising:
administering to the living system an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows: 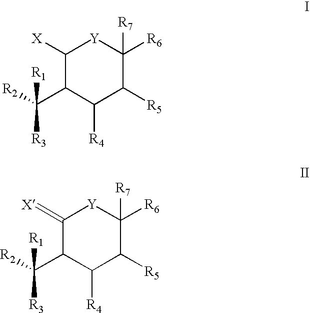
wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R₆ is H, CH₃, CH₂CH₃, or CH₂CH₂CH₃, R₇ is H or CH₃, X is OH, SH, or NH₂, X′ is O, S, or NH, and Y is O, S, NH, or CH₂, under conditions effective to treat cancer.

16. The method according to claim 15, wherein the 11β-hydroxysteroid dehydrogenase is isoform I.

17. The method according to claim 15, wherein the 11β-hydroxysteroid dehydrogenase is isoform II.

18. The method according to claim 15, wherein the inhibitor has the formula:

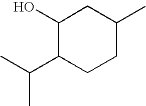

19. The method according to claim 15, wherein the inhibitor has the formula:

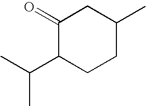

20. The method according to claim 15, wherein the administering is carried out topically.

21. The method according to claim 15, wherein the inhibitor is administered with pharmaceutically acceptable carrier, excipient, or stabilizer.

22. A method of treating obesity, diabetes mellitus, or a metabolic syndrome involving 11β-hydroxysteroid dehydrogenase in a living system comprising:
administering to the living system an inhibitor of cortisol-to-cortisone conversion, as mediated by 11β-hydroxysteroid dehydrogenase, of formula I or an inhibitor of cortisone-to-cortisol conversion, as mediated by 11β-hydroxysteroid dehydrogenase, of formula II or derivatives thereof as follows: 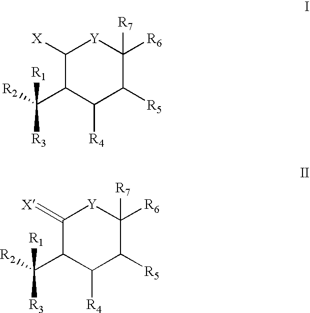
wherein R₁ is H or CH₃, R₂ is H, CH₃, or CH₂CH₃, R₃ is H, CH₃, CH₂CH₃, or CH₂CH₂CH₃, R₄ is H, CH₃, or CH₂CH₃, R₅ is H, CH₃, or CH₂CH₃, R₆ is H, CH₃, CH₂CH₃, or CH₂CH₂CH₃, R₇ is H or CH₃, X is OH, SH, or NH₂, X′ is O, S, or NH, and Y is O, S, NH, or CH₂, under conditions effective to treat obesity, diabetes mellitus, or a metabolic syndrome involving 11β-hydroxysteroid dehydrogenase.

23. The method according to claim 22, wherein the 11β-hydroxysteroid dehydrogenase is isoform I.

24. The method according to claim 22, wherein the 11β-hydroxysteroid dehydrogenase is isoform II.

25. The method according to claim 22, wherein the inhibitor has the formula:

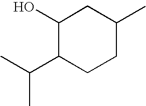

26. The method according to claim 22, wherein the inhibitor has the formula:

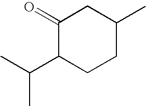

27. The method according to claim 22, wherein the administering is carried out topically.

28. The method according to claim 22, wherein the inhibitor is administered with pharmaceutically acceptable carrier, excipient, or stabilizer.

29. A method of inhibiting 11β-hydroxysteroid dehydrogenase in a living system comprising:
administering to the living system an effective amount of an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows: 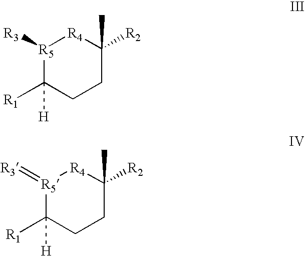
wherein R₁ is 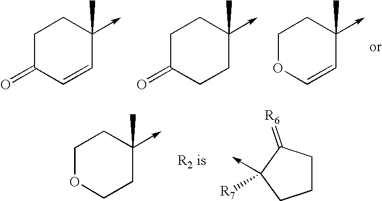
wherein R₆ is O or S and R₇ is H, OH, or halogen, or 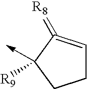
wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂.

30. The method according to claim 29, wherein the 11β-hydroxysteroid dehydrogenase is isoform I.

31. The method according to claim 29, wherein the 11β-hydroxysteroid dehydrogenase is isoform II.

32. The method according to claim 29, wherein the administering is carried out topically and/or systemically.

33. The method according to claim 29, wherein the inhibitor is administered with pharmaceutically acceptable carrier, excipient, or stabilizer.

34. A method of treating an inflammatory or allergic condition in a living system comprising:
administering to the living system an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows: 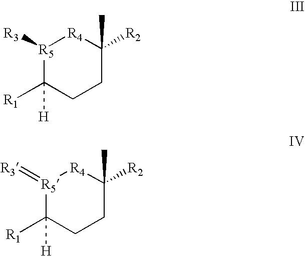
wherein R₁ is 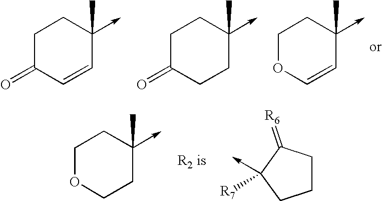
wherein R₆ is O or S and R₇ is H, OH, or halogen, or 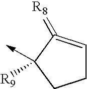
wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂, under conditions effective to treat an inflammatory or allergic condition.

35. The method according to claim 34, wherein the 11β-hydroxysteroid dehydrogenase is isoform I.

36. The method according to claim 34, wherein the 11β-hydroxysteroid dehydrogenase is isoform II.

37. The method according to claim 34, wherein the administering is carried out topically and/or systemically.

38. The method according to claim 34, wherein the inhibitor is administered with pharmaceutically acceptable carrier, excipient, or stabilizer.

39. A method of treating cancer in a living system comprising:
administering to the living system an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows: 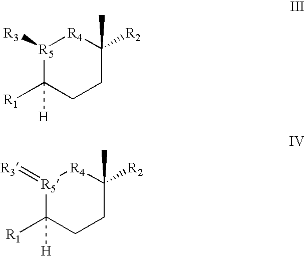
wherein R₁ is 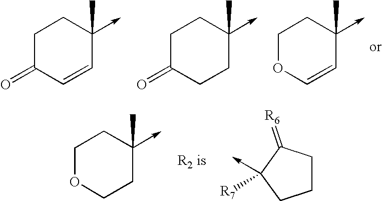
wherein R₆ is O or S and R₇ is H, OH, or halogen, or 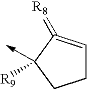
wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂, under conditions effective to treat cancer.

40. The method according to claim 39, wherein the 11β-hydroxysteroid dehydrogenase is isoform I.

41. The method according to claim 39, wherein the 11β-hydroxysteroid dehydrogenase is isoform II.

42. The method according to claim 39, wherein the administering is carried out topically and/or systemically.

43. The method according to claim 39, wherein the inhibitor is administered with pharmaceutically acceptable carrier, excipient, or stabilizer.

44. A method of treating obesity, diabetes mellitus, or a metabolic syndrome involving 11β-hydroxysteroid dehydrogenase in a living system comprising:
administering to the living system an inhibitor of cortisol-to-cortisone conversion, as mediated by 11β-hydroxysteroid dehydrogenase, of formula III or an inhibitor of cortisone-to-cortisol conversion, as mediated by 11β-hydroxysteroid dehydrogenase, of formula IV or derivatives thereof as follows: 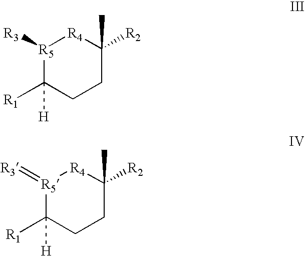
wherein R₁ is 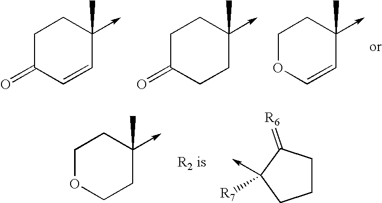
wherein R₆ is O or S and R₇ is H, OH, or halogen, or 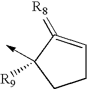
wherein R₈ is H, OH, or halogen, and R₉ is H, OH, or halogen, and R₃ is OH, SH, or NH₂, R₃′ is O, S, or NH, R₄ is O, S, NH, or CH₂, R₅ is N or CH₂, and R₅′ is SO or CH₂, under conditions effective to treat obesity, diabetes mellitus, or a metabolic syndrome involving 11β-hydroxysteroid dehydrogenase.

45. The method according to claim 44, wherein the 11β-hydroxysteroid dehydrogenase is isoform I.

46. The method according to claim 44, wherein the 11β-hydroxysteroid dehydrogenase is isoform II.

47. The method according to claim 44, wherein the administering is carried out topically and/or systemically.

48. The method according to claim 44, wherein the inhibitor is administered with pharmaceutically acceptable carrier, excipient, or stabilizer.

49. A method of inhibiting 11β-hydroxysteroid dehydrogenase in a living system comprising:
administering to the living system an effective amount of an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows: 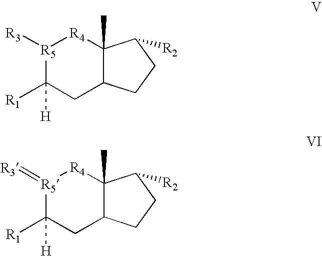
wherein R₁ is 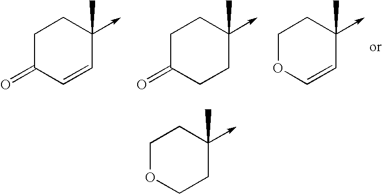
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

50. The method according to claim 49, wherein the 11β-hydroxysteroid dehydrogenase is isoform I.

51. The method according to claim 49, wherein the 11β-hydroxysteroid dehydrogenase is isoform II.

52. The method according to claim 49, wherein the administering is carried out topically and/or systemically.

53. The method according to claim 49, wherein the inhibitor is administered with pharmaceutically acceptable carrier, excipient, or stabilizer.

54. A method of treating an inflammatory or allergic condition in a living system comprising:
administering to the living system an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows: 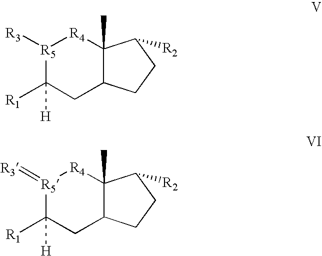
wherein R₁ is 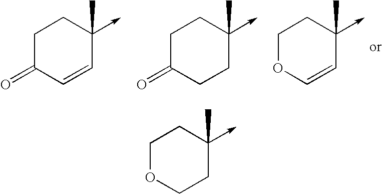
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat an inflammatory or allergic condition.

55. The method according to claim 54, wherein the 11β-hydroxysteroid dehydrogenase is isoform I.

56. The method according to claim 54, wherein the 11β-hydroxysteroid dehydrogenase is isoform II.

57. The method according to claim 54, wherein the administering is carried out topically and/or systemically.

58. The method according to claim 54, wherein the inhibitor is administered with pharmaceutically acceptable carrier, excipient, or stabilizer.

59. A method of treating cancer in a living system comprising:
administering to the living system an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows: 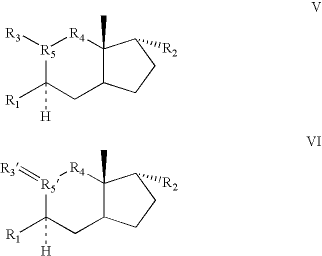
wherein R₁ is 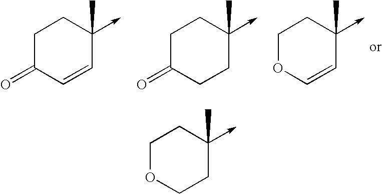
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat cancer.

60. The method according to claim 59, wherein the 11β-hydroxysteroid dehydrogenase is isoform I.

61. The method according to claim 59, wherein the 11β-hydroxysteroid dehydrogenase is isoform II.

62. The method according to claim 59, wherein the administering is carried out topically and/or systemically.

63. The method according to claim 59, wherein the inhibitor is administered with pharmaceutically acceptable carrier, excipient, or stabilizer.

64. A method of treating obesity, diabetes mellitus, or a metabolic syndrome involving 11β-hydroxysteroid dehydrogenase in a living system comprising:
administering to the living system an inhibitor of cortisol-to-cortisone conversion, as mediated by 11β-hydroxysteroid dehydrogenase, of formula V or an inhibitor of cortisone-to-cortisol conversion, as mediated by 11β-hydroxysteroid dehydrogenase, of formula VI or derivatives thereof as follows: 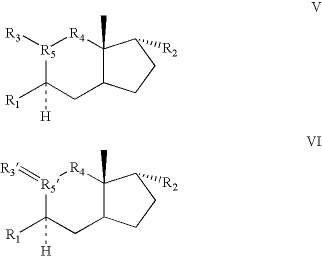
wherein R₁ is 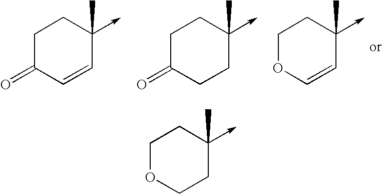
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat obesity, diabetes mellitus, or a metabolic syndrome involving 11β-hydroxysteroid dehydrogenase.

65. The method according to claim 64, wherein the 11β-hydroxysteroid dehydrogenase is isoform I.

66. The method according to claim 64, wherein the 11β-hydroxysteroid dehydrogenase is isoform II.

67. The method according to claim 64, wherein the administering is carried out topically and/or systemically.

68. The method according to claim 64, wherein the inhibitor is administered with pharmaceutically acceptable carrier, excipient, or stabilizer.

69. A method of inhibiting 11β-hydroxysteroid dehydrogenase in a living system comprising:
administering to the living system an effective amount of an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows: 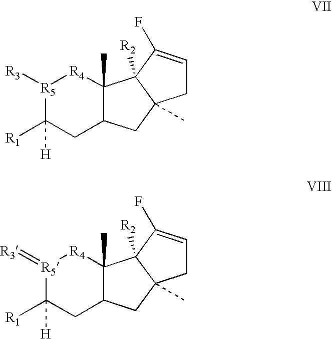
wherein R₁ is 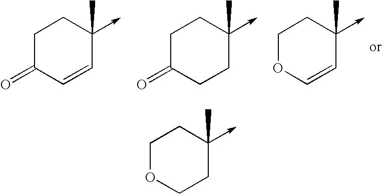
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂.

70. The method according to claim 69, wherein the 11β-hydroxysteroid dehydrogenase is isoform I.

71. The method according to claim 69, wherein the 11β-hydroxysteroid dehydrogenase is isoform II.

72. The method according to claim 69, wherein the administering is carried out topically and/or systemically.

73. The method according to claim 69, wherein the inhibitor is administered with pharmaceutically acceptable carrier, excipient, or stabilizer.

74. A method of treating an inflammatory or allergic condition in a living system comprising:
administering to the living system an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows: 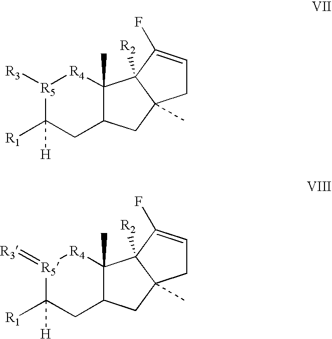
wherein R₁ is 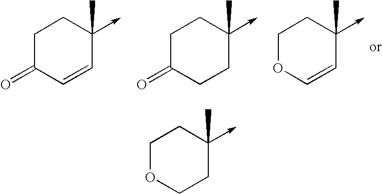
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat an inflammatory or allergic condition.

75. The method according to claim 74, wherein the 11β-hydroxysteroid dehydrogenase is isoform I.

76. The method according to claim 74, wherein the 11β-hydroxysteroid dehydrogenase is isoform II.

77. The method according to claim 74, wherein the administering is carried out topically and/or systemically.

78. The method according to claim 74, wherein the inhibitor is administered with pharmaceutically acceptable carrier, excipient, or stabilizer.

79. A method of treating cancer in a living system comprising:
administering to the living system an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows: 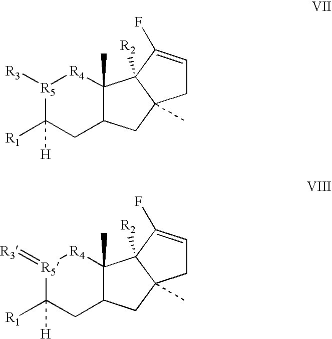
wherein R₁ is 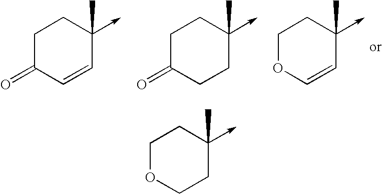
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat cancer.

80. The method according to claim 79, wherein the 11β-hydroxysteroid dehydrogenase is isoform 1.

81. The method according to claim 79, wherein the 11β-hydroxysteroid dehydrogenase is iso form II.

82. The method according to claim 79, wherein the administering is carried out topically and/or systemically.

83. The method according to claim 79, wherein the inhibitor is administered with pharmaceutically acceptable carrier, excipient, or stabilizer.

84. A method of treating obesity, diabetes mellitus, or a metabolic syndrome involving 11β-hydroxysteroid dehydrogenase in a living system comprising:
administering to the living system an inhibitor of cortisol-to-cortisone conversion, as mediated by 11β-hydroxysteroid dehydrogenase, of formula VII or an inhibitor of cortisone-to-cortisol conversion, as mediated by 11β-hydroxysteroid dehydrogenase, of formula VIII or derivatives thereof as follows: 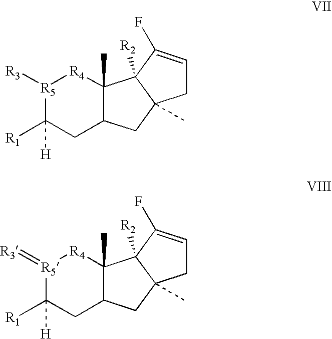
wherein R₁ is 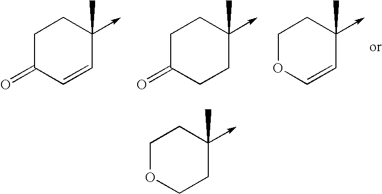
R₂ is H, OH, or halogen, R₃ is OH, SH, or NH₂, R₃′ is O, S, or NH, R₄ is O, S, NH, or CH₂, R₅ is N or CH₂, and R₅′ is SO or CH₂, under conditions effective to treat obesity, diabetes mellitus, or a metabolic syndrome involving 11β-hydroxysteroid dehydrogenase.

85. The method according to claim 84, wherein the 11β-hydroxysteroid dehydrogenase is isoform I.

86. The method according to claim 84, wherein the 11β-hydroxysteroid dehydrogenase is isoform II.

87. The method according to claim 84, wherein the administering is carried out topically and/or systemically.

88. The method according to claim 84, wherein the inhibitor is administered with pharmaceutically acceptable carrier, excipient, or stabilizer.
